(19) United States
(12) Patent Application Publication
(10) Pub. No.: US 2014/0377843 A1
(43) Pub. Date: Dec. 25, 2014

(54) AUTOMATED MICROBIOLOGY LABORATORY INSTRUMENT AND SYSTEM USES THEREOF

(71) Applicant: King Abdulaziz City for Science and Technology, Riyadh (SA)

(72) Inventor: Essam Jamil Alyamani, Riyadh (SA)

(21) Appl. No.: 13/924,579

(22) Filed: Jun. 23, 2013

(51) Int. Cl. C12M 1/30

(52) U.S. Cl. CPC C12M 33/02
USPC 435/243; 435/289.1

(57) ABSTRACT

The present application discloses an automated laboratory system comprising of an instrument and an accompanied system to prepare biological culture plates specifically microbiological culture plates for sample inoculation and a process of sample inoculation. Further, the application discloses various modules of a system required to run the different units of the instrument.

### FIELD OF TECHNOLOGY

The present disclosure generally relates to an automated laboratory instrument and its system for laboratory work. More specifically, the present invention relates to the general microbiology practice in the laboratory to automate some functions such as filling petri dishes.

### BACKGROUND

Routine microbiological methods include preparing media, sterilization of culture plates or using pre-sterilized culture plates, pouring media into the culture plates, incubating the culture plates in a separate incubator, inoculating the plates with the required sample and incubating the plates in required culture growth conditions. Each and every step in the routine microbiological procedure requires a separate instrumentation and a separate space area which increases the chances of sample and plate contamination. Researchers use a number of aseptic techniques and other good microbiological practices to prevent contamination both to the individual working in the laboratory and to the microbiological sample.

As such a standard microbiology laboratory carries laminar flow cabinets, autoclave, colony counter, incubator, vaccine bath, inoculation loop, gas-pak, vacuum pump, desiccator, microscope among other apparatus depending on the experiments being conducted in a particular laboratory. Cost of purchasing and maintaining these instruments and overall cost of the laboratory can thus be very high. Also microbiology laboratory needs very high maintenance to prevent contamination while performing culture plate preparation and sample inoculation.

Further, microbiology laboratory also require trained individuals competent enough to handle microbial material and to work with microorganisms. There is no one solution where both the professionals and non-professionals can work with microbiological samples in both small and large settings while also maintaining the aseptic conditions and have less processing time.

### SUMMARY

The invention relates to the general practice of culture plate preparation and sample inoculation done routinely in microbiology laboratories worldwide. The invention also relates to an automated biology laboratory setting such as microbiology laboratory setting.

In one embodiment, an instrument and a system to run the instrument as disclosed in the present application is used to prepare the biological cultures by one touch of a button. In another embodiment, an instrument and a system to run the instrument as disclosed in the present application is used to prepare the cell cultures to study by one touch of a button. In various embodiments, an instrument and a system to run the instrument as disclosed in the present application is used to prepare the microbiological colonies by one touch of a button.

In one embodiment, the said instrument as disclosed in the present application is divided into different units to perform a step-wise preparation of biological culture plates for sample inoculation and the sample inoculation. In another embodiment, the said instrument as disclosed in the present application comprise of a single continuous chamber to carry a process of preparing biological culture plates for sample inoculation and sample inoculation.

In one embodiment, the said instrument comprises of a receiving unit 102, a media dispensing unit 104, a shaking unit 106, an incubation/inoculation/inoculation unit 108, a coding unit 110, a sample receiving unit 112, an irradiation unit 114, a sample inoculation and streaking unit 116, a growth unit 118, a belt running within the instrument 120, and a user control unit 124. The said instrument further comprises of an intake window 101 and an outtake window 126 to load and unload the culture plates from the said instrument. The said instrument may further comprise of a culture plate warmer depending on experimental needs. In one embodiment, the said instrument assembly is in a glass box cover 122 or other suitable material casing to perform the experiment in compact settings and also to maintain aseptic conditions while working on the said instrument. Further, a user can also view the progress of culture plates from outside while the running of the said instrument. In one embodiment, a belt 120 moves within the system to carry culture plates from one unit to another during the preparation of biological culture plates for sample inoculation and sample inoculation. In another embodiment, a stainless steel belt moves within the system to carry culture plates from one unit to another while preparing biological culture plates for sample inoculation and sample inoculation. In various embodiments, a rack capable of holding culture plates of various sizes moves within the system to carry culture plates from one unit to another while preparing biological culture plates for sample inoculation and sample inoculation.

In one embodiment, a system to run the said instrument comprises of a media dispensing module 202, an incubator control module 204, a wireless module 206, a movement control module 208, a bar code module 210, an irradiation control module 212, a user control module 214, a sample management module 216, a plate dispenser module 218 and an inoculation module 220. In one embodiment, a system to run the said instrument comprises of a tube dispenser module, sensor control module and a digital display module among other modification done within a system to make it more user friendly.

In one embodiment, the said instrument comprises of a microbiological culture plates receiving unit 102 wherein microbiological culture plates of different sizes can be received into the said instrument depending on the experiment needs. In another embodiment, the said instrument comprises of a microbiological culture tubes receiving unit whereby microbiological culture tubes of different sizes and volume are received into the said instrument. In various embodiments, the said instrument comprises of an apparatus receiving unit whereby apparatus to be used to biological culture preparation can be received into the said instrument. In one embodiment, plate dispenser module 218 of the said system contains information for smooth running of the receiving unit 102 of the said instrument. Information related to the number of plates, volume and size of plates, number of plates to be dispensed for a particular experiment and other related information is present in the plate dispenser module 218 of the said system.

In one embodiment, culture plates or other apparatus to be used according to the experiment are sterilized before loading onto the said instrument like pre-sterilized apparatus commercially available whereas in another embodiment, culture plates or other apparatus is sterilized after loading onto the said instrument.

In one embodiment, a media is prepared for the cultivation of biological sample. In another embodiment, a media is prepared for the cultivation of microbiological sample. Media to be used in the said instrument can be either differential or selective or can be both depending on the experimental needs. Antibodies or other compounds may also be added into the media before it is dispensed into the culture plates. In one embodiment, a required volume of media is filled in a sterilized tank and assembled by connecting to the said instrument from outside whereas in another embodiment, small batches of pre-determined volume of media are introduced into the said instrument.

In one embodiment, the said instrument comprises of a media dispensing unit 104 which works in coordination with media dispensing module 202 wherein a pre-determined volume of media is poured into the culture plates. In another embodiment, varying volume of media is poured into the culture plates depending on their size, volume and experimental needs. In one embodiment, instructions and information for media dispensing unit 104 is present in media dispensing module 202 of the said system. In another embodiment, information regarding volume of media to be dispensed, type of media to be used, temperature at which the media is to be maintained is present in the media dispensing module 202 of the said system. The media dispensing module 202 also contain programs with pre-stored values for routinely used microbiological media and a user can just select a program from the list according to experimental needs.

In one embodiment, a plate shaker is present within the said instrument to gently shake the culture plates which helps in even distribution of media in culture plates. In another embodiment, a belt is moved such that it results in even distribution of media in culture plates.

In one embodiment, incubator present in the incubation/inoculation unit 108 is set ON at a pre-selected temperature before the culture plate enters into the incubation/inoculation unit 108. In another embodiment, incubator is set ON at a pre-select temperature before media is dispensed into the culture plates. The incubator is set at a fixed temperature either to solidify the media in culture plates or to keep the media in gel or liquid state depending on the media used and the experimental needs. In one embodiment, the temperature requirements according to the media being used are present in the incubator control module 204 of the said system of the said instrument. In another embodiment, the temperature requirements depends on the media being used and may be then feed into the incubator control module 204 of the said system by the user or selected from stored programs before the start of the said instrument.

In one embodiment, the culture plates are labeled with a unique bar code in the coding unit 110 of the said instrument to identify each and every culture plate. In another embodiment, culture plates are labeled manually with a unique number or other identification code depending on users comfort before loading into the receiving unit 102 of the said instrument. The bar code is then scanned in the coding unit 110 of the said instrument which is the first step of quality control process of the said instrument. The information regarding unique bar code given to the culture plates and to the samples under investigation is stored into the bar code module 210 of the said system. The bar code module 210 matches the code of the culture plates with the sample code. Culture plates are always scanned as they pass the coding unit 110 of the said instrument and the information is passed to the bar code module of the said instrument wherein the information is processed for quality control.

In one embodiment, the said system has a movement control module 208 to maintain and coordinate the movement of culture plates within the said instrument. In another embodiment, the said system has a movement control module 208 to maintain and coordinate the movement of an intake window 101, an outtake window 126, a shaking unit 106, a belt 120 moving within the instrument, movement of sample in and out of the said instrument, movement of media tank in and out of the said instrument, movement of inoculating needles, loops, movement of incubator doors among other movements happening within the said instrument.

In one embodiment, inoculating needles are used for inoculation and streaking of the sample onto the media filled culture plates. In another embodiment, inoculating loops are used for inoculation and streaking of the sample onto the media filled culture plates. In one embodiment, inoculating needles are sterilized with exposure to UV light source before picking the sample for inoculation and streaking onto the media filled culture plates. In one embodiment, a single inoculating needle or loop can be used for inoculating a number of samples and the inoculating needle or loop is then sterilized every time to prevent contamination. In another embodiment, disposable inoculating needle or tip can be used for inoculating samples into the media filled culture plates. In one embodiment, a pre-sterilized needle or loop is loaded into the said instrument. In another embodiment, needle or loop is sterilized just before inoculating the sample. In various embodiments, a pre-sterilized needle or loop is loaded onto the said instrument and is sterilized again by exposing the needles/loops to UV light. Sterilization of inoculating needles is done to maintain aseptic working conditions. In one embodiment, sterilization of inoculating needles/loop is done by exposing them to UV light for a specific period of time in the said instrument. In another embodiment, sterilization of inoculating needles/loop is done by exposing them to UV light for a specific time in the irradiation unit 114 of the said instrument. In one embodiment, irradiation control module 212 of the said system carries all the information required for smooth running of the irradiation unit. In another embodiment, irradiation control module 212 of the said system carries information such as source of UV and time of UV exposure.

In one embodiment, a pre-determined volume of sample is inoculated into the media filled biological culture plates. In another embodiment, a pre-determined volume of sample is inoculated and streaked into the media filled microbiological culture plates. In various embodiments, every media filled culture plates may be inoculated with a different volume of sample depending on the experimental setup. In one embodiment, volume of sample may be entered into the sample management module 216 of the said system. In one embodiment, sample management module 216 works in coordination with irradiation control module 212 and inoculation module 220 to run the desired experiment smoothly. In another embodiment, sample management module 216 works in coordination with various other modules of the said system. In one embodiment, sample management module 216 can store information such as type of sample, nature of sample, number of samples, volume of sample to be used in inoculation along with other related information regarding the sample that a user can feed into the system depending on the experimental needs. In another embodiment, values are pre-entered in the sample management module 216 and inoculation module 220. In another embodiment, a user may enter values in the sample management module 216 and inoculation module 220 to run the designed experiment. In one embodiment, sample used can be a clinical sample or a non-clinical sample. In another embodiment, sample used can be a microbiological sample or cells.

In one embodiment, culture plates are moved to a pre-warm incubator in the growth unit 118 following inoculation for the growth of biological culture. In another embodiment, culture plates are moved to a pre-warm incubator in the growth unit 118 following inoculation for the growth of microbiological colonies. In one embodiment, incubator is set ON at a required temperature just before culture plates enter the growth unit 118 and the culture plates are left into the incubator for a time sufficient for the growth of biological cultures or cell cultures. In another embodiment, incubator is set ON at a required temperature just before culture plates enter the growth unit and the culture plates are left into the incubator for a time sufficient for the growth of microbiological colonies. In one embodiment, incubator control module 204 of the said system manages the information regarding temperature, percentage of CO₂ among other growth conditions for the incubator present in the growth unit 118 of the said instrument. A user can also feed the temperature and values for other parameters in the incubator control module 204 of the said system depending on different experimental needs or can use the pre-entered values already present in standard programs feed present in the incubator control module 204 of the said system.

In one embodiment, a belt 120 runs within the said instrument and is a way of communication between different units. In another embodiment, a belt 120 has a number of sensors attached to it on both sides. In another embodiment, a belt has a number of sensors present within the belt. Activity recorded from the sensors helps in monitoring the movement of culture plates within the said instrument and also make sure that culture plates are moving in the desired unit and direction within the said instrument. The activities and readouts from the sensors present on the sides of the belt can be monitored through the movement control module 208 of the said system. A user can also enter information such as speed of the belt, the direction of the movement of the belt and other related information in the movement control module 208 of the said system. A user may also select the parameters from already fed programs present within the said system.

In one embodiment, a microscope may also be placed after the growth unit 118 within the said instrument to monitor the growth of biological culture in culture plates. In another embodiment, a microscope may be attached from outside of the said instrument to monitor the growth of biological culture in culture plates. In one embodiment, the screen of a computer or a laptop or other digital device may be used to monitor the outcome of culture growth. In another embodiment, culture plates can also be viewed under the microscope present separately from the said instrument.

In one embodiment, a user working on the said instrument works through the user control module 214 of the said system to synchronize the system for smooth functioning of the said instrument. In one embodiment, a user working on the said instrument works through the already fed program sets in user control module 214 of the said system. In various embodiments, a user working on the said instrument feed values for different parameters like media preparation, culture dishes preparation, temperature of incubation/inoculation, type of sample, volume of sample to be used in inoculation among others information through user control module 214 of the said system. In one embodiment, the said system comprises at least one workstation with software comprising of the said system.

In one embodiment, a user may run the system for a full-run task or half-run task. Full-run task comprises of loading culture plates, dispensing the media in culture plates in media dispensing unit 104, setting the media in incubator in incubation/inoculation unit 106, loading the samples into sample receiving unit 112, irradiating the inoculating needles or loops in the irradiation unit 114, inoculating and streaking the culture plates with the sample under investigation in the sample inoculating and streaking unit 116, and growing the biological culture or microbiological colonies by placing the culture dishes into the incubator present in the growth unit 118. Half-run task comprises of irradiating the inoculating needles or loops in the irradiation unit 114 and inoculating the pre-prepared media filled culture plates with the sample under investigation in the sample inoculating and streaking unit 116 and growing the sample colonies by placing the culture dishes into the incubator present in the growth unit 118.

In one embodiment, steps for culture plate preparation and growth of biological culture in an enclosed instrument under aseptic conditions are provided. In another embodiment, steps for microbiological culture plate preparation and growth of microbiological colonies in an enclosed instrument under aseptic conditions are provided.

In one embodiment, a process of preparing culture plates and sample inoculation is disclosed wherein a user may load culture plate into the receiving area 102 through an intake window 101, media to be used in a media tank in the media dispensing unit 104, samples under investigation through an intake window 101 into sample receiving area 110, inoculating needles or loops into the sample inoculating and streaking unit 114 before pushing the start button to ON of the said instrument. In another embodiment, a process of preparing culture plates and sample inoculation is disclosed wherein a user may load culture dishes into the receiving area 102 through an intake window 101, media to be used in a media tank in the media dispensing unit 104, samples under investigation through an intake window 101 into sample receiving area 110, inoculating needles or loops into the sample inoculating and streaking unit 114 after pushing the start button to ON of the said instrument. In one embodiment, steps to prepare culture plates and sample inoculation comprise of entering the sample code into the said system, selecting the conditions to run the experiment from the programs already fed into the said system, loading the culture plates into the receiving area 102, loading the media to be used in a media tank in media dispensing unit 104, loading the samples under investigation through intake window, pressing the start button to ON present outside of the said instrument.

In one embodiment, the said instrument is sterilized by exposing it to UV light after pushing the start button to ON which will sterilize all the units of the said instrument as such before a user runs the experiment and will thus ensure aseptic working conditions. In another embodiment, the said instrument is sterilized by exposing it to UV light after pushing the start button to OFF at the end of the experiment which will ensure instrument long-life and maintenance.

In one embodiment, a process of preparing culture plates and sample inoculation is disclosed wherein steps include loading the required number of culture plates in the receiving unit 302, 304, loading the media being used in the media tank 306, loading the samples under investigation in sample receiving unit 124, pushing the start button to ON, moving the culture plates on belt into the media dispensing unit 104 and filling the culture plates with required volume of media 308, gently shaking the culture plates, solidifying the media by moving the culture plates on belt into the incubator present within the incubation/inoculation unit 106, moving solid media containing culture plates 310 into the coding unit, bar coding the solidifies culture plates with a unique code 312, irradiating the inoculating tip 314 in irradiation unit 114, picking up the sample with irradiated inoculated tip 316 from sample receiving area 112, inoculating and streaking the sample into coded culture plates 318 by moving the culture plates on belt to sample inoculation and streaking unit 116, incubating the culture plates with sample into incubator 320 present in the growth unit 118 for biological culture growth or growth of microbiological colonies.

In another embodiment, the general steps of culture plate preparation and sample inoculation within the said instrument comprises of pushing the start button to ON 401, preparing the media for dispensing 402, sterilizing the culture plates 404, filling the culture plates with required amount of media and coding the culture plates 406, coding the samples under investigation 408, sterilizing the inoculating tips and inoculating the sample on culture plates 410, growing the inoculated samples in culture plates 412, monitoring the growth of culture or microbiological colonies 414.

Other features will be apparent from the accompanying figures and from the detailed description that follows.

### BRIEF DESCRIPTION OF THE DRAWINGS

Example embodiments are illustrated by way of examples and no limitation in the tables and in the accompanying figures, like references indicating similar elements and in which:

FIG. 1 shows a block diagram representation of an instrument as disclosed.

FIG. 2 shows a block diagram representation of a system as disclosed.

FIG. 3 shows culture plate movement within the instrument.

FIG. 4 shows the steps involved in preparing the culture plates for sample inoculation and sample inoculation and streaking.

Other features of the present embodiments will be apparent from the accompanying figures and from the detailed description that follows.

### DETAILED DESCRIPTION

Several embodiments of the presently disclosed application describe an automated laboratory system comprising of an instrument and an accompanied system to perform culture plates preparation and sample inoculation. Further, the application discloses various modules of a system required to run the instrument. Although the present embodiments have been described with reference to specific example embodiments, it will be evident that various modifications and changes may be made to these embodiments without departing from the broader spirit and scope of the various embodiments.

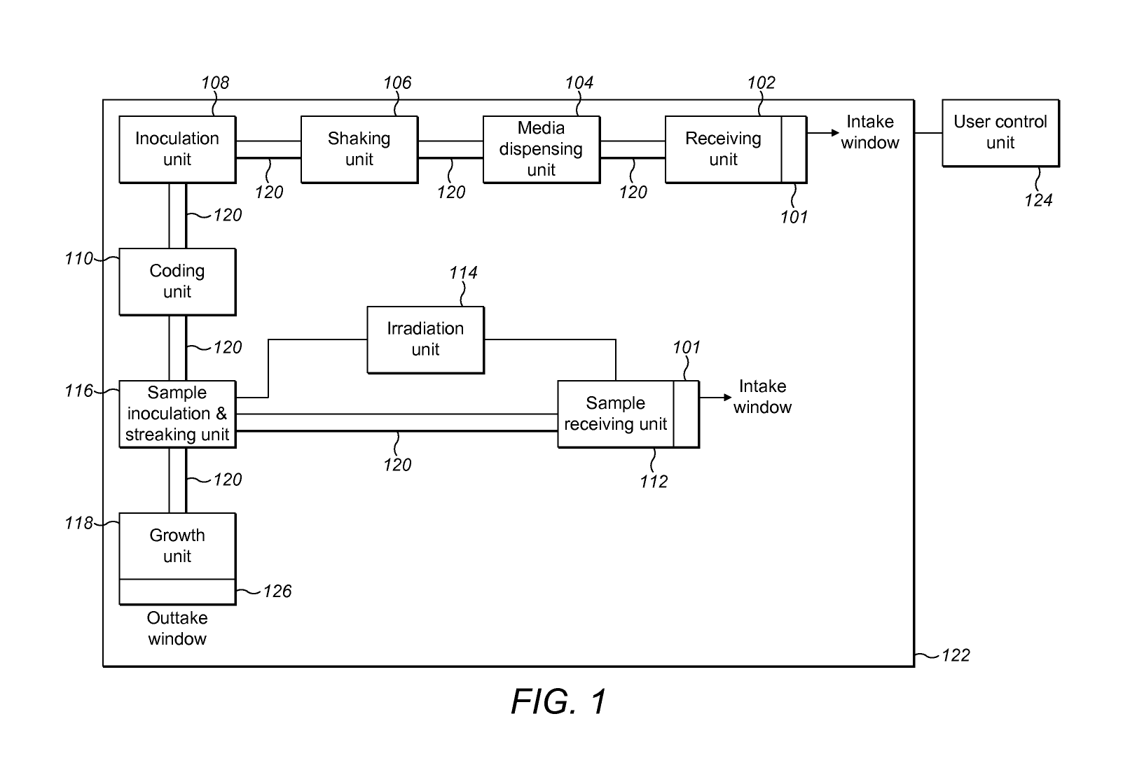

FIG. 1 shows different units of an instrument used in preparing biological culture plates and sample inoculation. The instrument comprises of an intake window 101, a receiving unit 102, a media dispensing unit 104, a shaking unit 106, an incubation/inoculation unit 108, a coding unit 110, a sample receiving unit 112, an irradiation unit 114, a sample inoculation and streaking unit 116, a growth unit 118, a belt running within the instrument 120, an outtake window 126, a box cover on the said instrument 122 and a user control unit 124. The intake window as disclosed above open up to place required number of culture plates into the receiving unit 102. The culture plates can be placed as a stack or a single culture plate at one time which depends on the experimental requirements. The intake window can be opened manually by a user, by a push button present outside or by simply giving a command from the said system connected to the said instrument. The receiving unit 102 is further connected to a media dispensing unit 104 through a belt 120 running within the said instrument. The belt aid in moving culture plates from one unit to another within the said instrument. Belt of any suitable material can be used within the instrument. In most embodiments, a stainless steel belt will be used within the said instrument.

Media in pre-determined volume is poured into the culture plates once they move into the media dispensing unit 104. Media dispensing unit 104 is further connected to a shaking unit 106. Culture plates are gently shaken properly to evenly distribute the media in the culture plates before they enter the incubation/inoculation unit 108. The culture plates are shaken by a shaker present in the shaking unit 106. Another method of evenly distributing the media is through the forward and reverse movement of belt after dispensing media in the culture plates. Media dispensing unit 104 further comprise of a removable media tank 306 with digitalized temperature settings and auto-pump dispenser for pouring media into the plates. The tank filled with the required media, more specifically agar gel can be introduced into the instrument before setting the instrument to ON or after setting the instrument to ON. The working of media dispensing unit 104 is further controlled via media dispensing module 202 present within the said system and carries information regarding different aspects such as type of media, temperature to store the media, total volume of media, antibodies and other known/unknown compounds to be added into the media. The volume of media to be dispensed also depends on the volume and size of culture plates or culture tubes being used in the instrument. A user may enter the volume of media to be dispensed or use the program already present in the system.

Culture plates filled with media are passed into the incubation/inoculation unit 108 to solidify the media or maintain the media in liquid/gel state and thus prepare the culture dishes for sample inoculation. Incubator in incubation/inoculation unit 108 is set ON at a pre-determined temperature before the culture plates filled with media enter. In most microbiological experiments, the temperature of the incubator will be set at 4° C. to solidify the media. Incubation/inoculation unit 108 is further connected to a coding unit 110 through running belt 120. Culture plates are given a unique code, the information for which is already present in the bar code module 210 of the said system. The unique code helps in identifying each and every sample with the results obtained and also each and every culture plate with the sample inoculated to maintain the accuracy of the experiment and results obtained. Coding unit is further connected to a sample inoculation and streaking unit 116. Sample inoculation and streaking unit 116 is further connected to sample receiving unit 112 and irradiation unit 114.

Irradiation unit 114 comprises of a UV light source to sterilize inoculating needles/loops present within the irradiation unit. One may also use pre-sterilized inoculating needles/loops and can be re-sterilized again to maintain highly aseptic working conditions. Inoculating needles/loops are sterilized in the irradiation unit for a specific time period before they perform the inoculation and streaking of the sample onto the media filled in the culture dishes. The UV sterilization of the needles/loops can be done simultaneously with the labeling of the culture plates in the coding unit of after the coding of the culture plates is complete and they enter the inoculating and streaking unit 116.

The sample receiving unit 112 has an intake window 101 which is open to receive the samples under investigation. The samples are labeled with a unique code to identify each and every sample being used in the experiment. Sample inoculation and streaking unit 116 comprises of inoculating needles/loops which are first exposed to UV light, then moved into the sample receiving unit 116 to pick a pre-determined volume of the sample and then enter into the inoculating and streaking unit to inoculate and then streak the sample onto media to evenly distribute the sample.

The culture plates are then moved into the growth unit 118 of the said instrument where the plates are left for a required period of time and under required conditions for the growth of biological cultures or microbiological colonies. The growth unit 118 comprise of an incubator maintained at a temperature required for the growth of microbiological or biological cultures. For most microbiological experiments, the temperature of the incubator in growth unit is set at 37° C. The temperature conditions of the incubator present in the growth unit is maintained by the incubator control module 204 of the said system. The instrument as such is covered by a glass box 122 which helps in maintaining aseptic conditions as well as to observe the progress of the culture plates within the instrument. The said instrument as such may also be covered by a plastic, metal or other suitable covering to maintain aseptic working conditions.

A belt 120 runs within the instrument and move the culture plates from one unit to another within the instrument. One may also use conveyor belts or other suitable material belt. The belt is equipped with a number of sensors present on both sides of the belt whose activity ensures that the system is running in the desired direction. Sensors may also be present within the belt or at various other points on the belt to monitor the activities of culture plates. The sensors are connected to the movement control module 208 of the system and therefore work in coordination with media dispensing module 202, incubator control module 204, wireless module 206, bar code module 210, irradiation control module 212, user control module 214, sample management module 216, plate dispenser module 218 and inoculation module 220. The activity from the sensors in continuously monitored and helps in tracking the movement of culture plates as they move along within the instrument up till outtake window. Activity from the sensors also help in quality control checks at various steps within the said instrument like discarding the poorly or over filled media culture plates, matching the bar codes on culture plates with that on samples, matching the manually labeled codes on culture plates as they pass the coding unit.

The instrument further may also comprise of a microscope to observe the culture growth or growth of microbiological colonies. The output from the microscopic observations can be viewed on a large screen of output device. The instrument is further connected to a user control unit 124 to control and input values for different factors and conditions for running various biological experiments especially microbiological experiments.

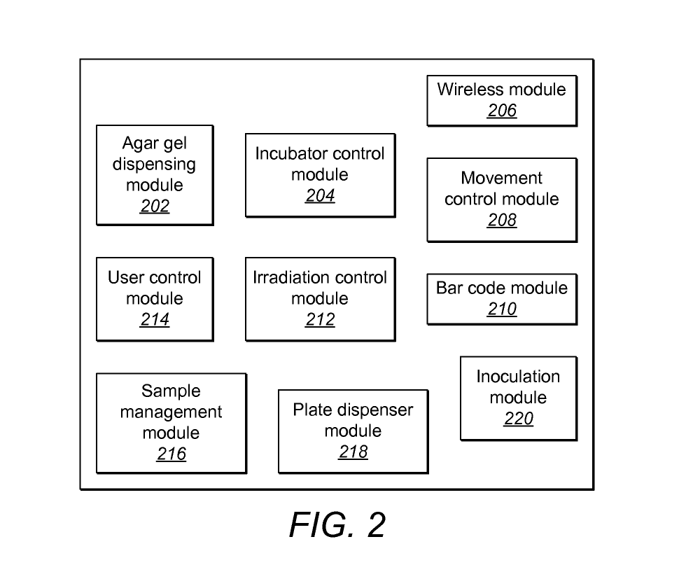

FIG. 2 shows different modules of a system connected to the instrument which is essential for the smooth functioning of the said instrument. The system 222 comprises of a media dispensing module 202, an incubator control module 204, a wireless module 206, a movement control module 208, a bar code module 210, an irradiation control module 212, a user control module 214, a sample management module 216, a plate dispenser module 218 and an inoculation module 220.

Media dispensing module 202 maintains the conditions and factors associated to the media being used for the biological culture growth. The module also contain information regarding temperature requirement to maintain the media in liquid, semi-liquid or gel state, storage conditions requirement, amount of media to be dispensed, information regarding mixing the media continuously, at regular intervals throughout the experiment or just before dispensing the media into culture plates, information related to antibodies or any other compound to be added in the media among other related information. It is pre-fed with the information which is standard for known media used commercially and non-commercially in biological and microbiological laboratories worldwide. It may contain information for blood agar media, eosin methylene blue agar media (EMB), macconkey agar (MAC), mannitol salt agar (MSA), milk agar, phenylethanol agar (PEA), sabourand dextrose agar (SDA), simmons citrate agar, spirit blue agar, trypticase soy agar (TSA) along with various other kind of media being used in the biological and microbiological laboratories worldwide. A user may choose the conditions and other factors from already stored programs or may enter information depending on the experimental needs and media used apart from the stored list. Incubator control module 204, maintains the incubator temperature present in the incubation/inoculation unit 108 and growth unit 118 of the said instrument. It contains information regarding the temperature required to solidify the media, maintain the media in liquid state or semi-liquid state depending on the experimental setup. In general incubator in incubation/inoculation unit is set at 4° C. and incubator in growth unit is maintained at 37° C. Further, the incubator control module 204 also helps in monitoring the rise and fall in the temperature of incubators while the experiment is running and also before the start of the experiment and after the experiment finishes.

Wireless module 206, helps in running the said instrument through a wireless mode. It makes the instrument usable in situations where the instrument is not connected to the system through cables or when a user chose to use Wi-Fi mode for communicating with the instrument. This also makes the instrument portable and thus can be used in remote settings, outdoor settings where a complete biological lab setup is not easy and possible. It also helps in making the instrument a part of mobile biological laboratory and can be used in space stations, desert locations, and other remote locations.

Movement control module 208 maintains all the different movements happening within the instrument. The said module helps in maintaining and coordinating the opening and closing of intake and outtake window, opening and closing of incubators present in the incubation/inoculation unit and the growth unit, movement of belt within the instrument in both forward and reverse direction, shaking of the plates to evenly distribute the media, movement of the culture plates from one unit to another within the instrument. Movement control module 208 works in coordination with media dispensing module 202, incubator control module 204, wireless module 206, bar code module 210, irradiation control module 212, user control module 214, sample management module 216, plate dispenser module 218 and inoculation module 220 to properly execute functioning of the said instrument and running of the experiment. Further, movement control module 208 also monitors the activities received from the sensors present on both sides of the belts to ensure belt is running in desired direction. The activity recorded from the sensors helps in quality control of the experiment as it will help in discarding the culture plates that do not meet the set standard for the experiment such as volume of media in every plate. If a less or more volume of media is dispensed into the culture plates than it will affect the weight of the culture plate onto the belt and thus the activities from the sensors will help in determining the weight of all the culture plates and will discard the culture plate which do not meet these standards.

Bar code module 210 maintains and stores information regarding the unique code given to each and every culture plate and to each biological sample (the word sample is used for a biological sample throughout the application). It helps in matching the culture plate code with the sample code and thus in quality control of the experiment.

Irradiation control module 212 maintains the conditions required to sterilize the instrument and inoculating needles/loops within the said instrument. It contains information such a source of UV being used, time of UV exposure, time for UV exposure. The irradiation control module 212 works in coordination with inoculation module 220, user control module 214 and movement control module 208 to maintain sterilized conditions within the said instrument. User control module 214 maintains all the inputs from user for different experimental conditions. A user may add any information pertaining to culture plates, media, incubation/inoculation temperature, sample being used, time of UV exposure, temperature to maintain growth conditions, speed of the belt, source of inoculation etc. User control module may also contain some in-built programs labeled as program 1, 2 and so on with standard experimental conditions used routinely in biological laboratories and user may select any program out of the list if chosen for. User control module works in coordination with media dispensing module 202, incubator control module 204, wireless module 206, bar code module 210, irradiation control module 212, sample management module 216, plate dispenser module 218 and inoculation module 220 for smooth running of the experiment and the instrument. User control module gives user an authority to control other modules and conditions for experiment.

Sample management module 216, works in coordination with sample receiving unit 112 and inoculation and streaking unit 116. It carries information such as type of sample, number of samples, codes given to various samples for identification, conditions to maintain the samples, volume of sample to be used in inoculation among other related information. It also acts as a check point for matching the codes on culture plates with the sample codes.

Plate dispenser module 218 maintain information on dispensing of the culture plates from the receiving unit 102, media dispensing unit 104, shaking unit 106, incubation/inoculation unit 108, coding unit 110, inoculation and streaking unit 116 and growth unit 118 of the instrument. It also carries information on the number of culture plates to be used in a particular experiment, type of culture plates, tubes or other appropriate apparatus used in the instrument. It helps in dispensing the culture plates onto the belt and also as the culture plates passes from one unit to another within the instrument.

Inoculation module 220 maintains all the information pertaining to medium of inoculation being used in the experiment. It can be inoculating needles, inoculating loops, inoculating tips or other inoculating apparatus depending on the availability and experimental needs. It also carries information for single use inoculating apparatus, re-usable inoculating apparatus and multi-use inoculating apparatus. It helps in monitoring the sample volume being used for inoculation which is generally followed by the streaking in case of microbiological samples.

The incubator control module 204 of the system helps in maintaining the temperature of the incubator present in the incubation/inoculation unit 106 of the instrument. The incubator may be set ON in parallel with switching the instrument ON and maintain the temperature of the incubator throughout the experimental run. Further, incubator can be set ON as soon as the culture plates reaches the media dispensing unit 104 so that the required temperature can be reach before the culture plates enter the incubation/inoculation unit 106. The functioning of incubator in the incubation/inoculation unit 106 is monitored and controlled by incubator control module 204 of the said system.

To start with, the system is set ON by pushing the start button on a desktop, a laptop or other digital device. Following this, the instrument is set ON by pushing the start button to ON present outside the instrument or through the user control module 214 of the system. The user control module 214 is present within a system 222 requires to run the instrument. A user control module 214 can be accessed through a computer loaded with the said system, a laptop loaded with the said system or any other digital device loaded with the said system. The user control module within the said system coordinates with the instrument through cables or through a wireless system. Once the system and instrument is set ON, the instrument with glass box cover ON is sterilized by exposing to UV for a required period of time. The UV light source may be located at the upper end of the glass box cover, lower end of the glass box cover or on the sides of the glass box cover. The time and amount of radiations at which the instrument is exposed is controlled via irradiation control module 212 present within the system to run the instrument. UV light is then turned OFF after sterilization process is complete. Culture plates are then loaded into the receiving unit of the instrument by opening of the intake window.

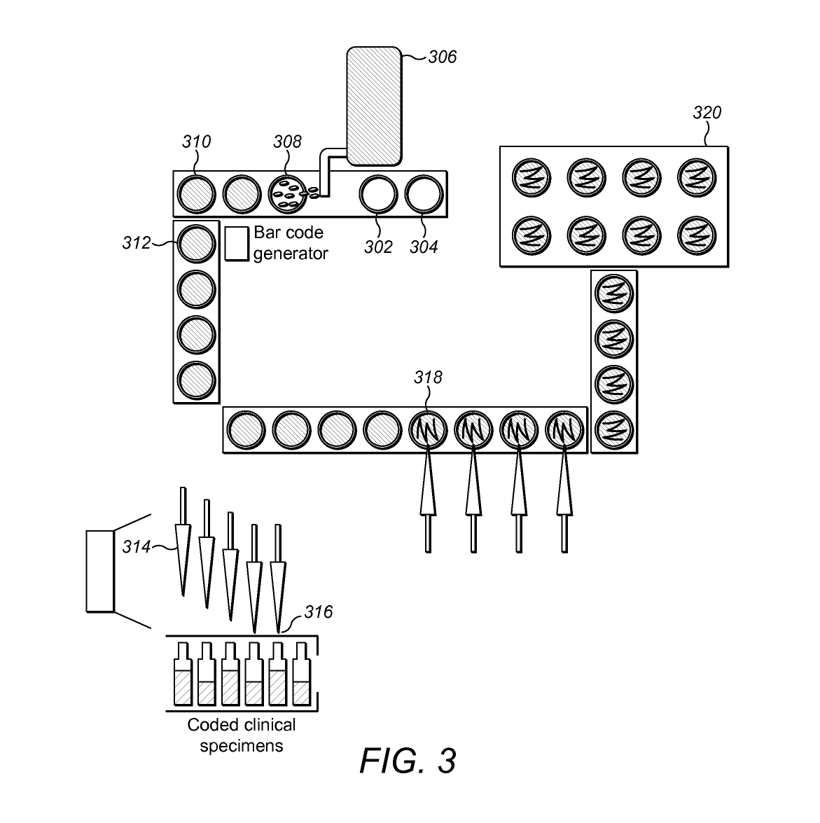

FIG. 3 shows the movement of culture plate within the said instrument during routine microbiological culture plate preparation and sample inoculation procedure. Culture plates 302, 304 are first received into the receiving unit 102 of the said instrument. The plate then enters into the media dispensing unit 104 via belt 120. A pre-determined volume of media is then dispensed from the auto-pump dispenser of the tank into the culture plates. Culture plates filled with media 308 is then moved into the incubation/inoculation unit 106 to solidify the media. In most experiments, incubator is set at a temperature of 4° C. to solidify the media but in other experiments it may be set at 37° C. to maintain the media at room temperature. Culture plate with solidified media 310, is then moved into the coding unit 108 of the said instrument where it is given a unique number for its identification. Culture plates can be given a unique code manually or electronically before they are loaded into the receiving unit of the said instrument. If labeled manually, culture plate code can then be entered into the bar code module and user control module of the said system. This will help in matching the culture plate code with the sample code and thus will aid in quality check. Labeled culture plate with solidified media 312 is then moved into the inoculation and streaking unit 114 of the said instrument. Sterilized inoculating needles/loops 314 then enter into the sample receiving area and pick a required volume of sample 316 and inoculate onto the solidified media containing culture plates 318. Inoculation is generally followed by streaking of the sample to ensure even distribution of the sample onto the media. The inoculated culture plates are then moved into the growth unit 116 of the instrument where they are left for a specific period of time 320.

Further, if the experiment demands monitoring the effect of antibodies, inhibitors, activators or test compounds on the growth of culture media then the system can be run in reverse direction up till inoculating and streaking unit 114 with the help of belt 118 controlled by movement control module 208 of the system. Test compounds/inhibitors/activators or other molecules are then introduced into the instrument through opening of intake window 101 of sample receiving unit 110. Inoculating needle is again sterilized as described above and a required volume of test compound is then introduced onto the culture plates with colonies. The culture plates will the again move to the growth unit for a specific time period to see the effect of test compound which can then be monitored under microscope or through other biological tests.

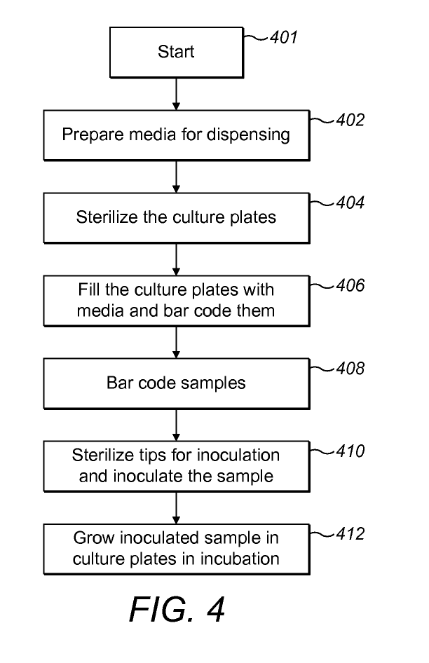

FIG. 4 shows the steps involved for preparing the culture plates and sample inoculation in the form of a flow chart. To start with instrument and system is set to ON 401. Media is then prepared for dispensing into the culture plates 402. Culture plates are then filled with a required volume of media followed by coding the culture plates with a unique code 404. Samples to be examined are also given a unique code 406 to identify them. Inoculating needles/loops are then sterilized by exposing them to UV for a prescribed time 408. Culture plates are inoculated with the sample followed by streaking 410. Culture plates are then introduced into the incubator to grow inoculated sample 412. The growth of inoculated samples are then monitored 414.

While the present disclosure has been described with reference to an exemplary embodiment, changes may be made within the purview of the appended claims, without departing from the scope and spirit of the present disclosure in its aspects. Also, although the present disclosure has been described herein with reference to particular materials and embodiments, the present disclosure is not intended to be limited to the particulars disclosed herein; rather, the present disclosure extends to all functionally equivalent structures, methods and uses, such as are within the scope of the instant claims. Accordingly, the specification and drawings are to be regarded in an illustrative rather than in a restrictive sense.

What is claimed is:

1. An instrument, comprising:
a receiving unit to receive a culture plate;
a media dispensing unit to dispense required volume of a media into the culture plate;
an incubation/inoculation unit to solidify the media in the culture plate;
a coding unit to bar code a solidified culture plate;
a sample inoculation to inoculate the sample onto the solidified culture plate;
a streaking unit to streak the sample that has been inoculated onto the solidified culture plate; and
a growth unit to provide a condition for the growth of biological culture plate for a designed experiment.

2. The instrument of claim 1, further comprising:
an intake window for introducing the culture plates into the said instrument;
an outtake window to remove the culture plates from the said instrument;
a shaking unit to gently shake the culture plates in the said instrument; and
an irradiation unit to sterilize the inoculating apparatus in the said instrument.

3. The instrument of claim 1, further comprising;
a belt running within the said instrument to carry the culture plate from one location to another.

4. The instrument of claim 1, further comprising:
a digital device comprising of a set of instructions to run the instrument.

5. The instrument of claim 1, wherein the receiving unit receives a culture tube.

6. The instrument of claim 2, wherein the instrument is in a compact assembly covered by a glass box.

7. The instrument of claim 4, wherein the set of instructions is in the form of various modules.

8. The instrument of claim 4, wherein the said instrument is used to prepare biological culture plates for inoculation and inoculating the sample.

9. The instrument of claim 1, wherein the said instrument is used to prepare microbiological colonies.

10. The instrument of claim 1, wherein the said instrument is used to prepare cell culture.

11. The instrument of claim 1, wherein an incubation/inoculation unit of said instrument maintains the media in at least one of a liquid and gel state.

12. A process, comprising:
loading required number of a culture plate in the receiving unit of an instrument;
dispensing required volume of a media in the culture plate using a media dispensing unit of the said instrument;
solidifying media in the culture plate in an incubator present in the incubation/inoculation unit of the said instrument;
coding the culture plate for identification in a coding unit of the said instrument;
inoculating the culture plate with a biological culture sample; and
growing the biological culture sample in a growth unit.

13. The process of claim 12, further comprising:
gently shaking the culture plates;
labeling and loading the samples in sample receiving unit of the said instrument; and
sterilizing the inoculating apparatus by exposing it to UV.

14. The process of claim 12, wherein a belt running to carry the culture plates from one unit to next within the said instrument.

15. A system, comprising:
a plate dispenser module to coordinate the movement of a culture plate in an instrument;
a media dispensing module to carry information and instruction for a media dispensing unit of the said instrument;
an incubator control module to carry information and instruction for an incubator in an incubation/inoculation unit and a growth unit of the said instrument;
a bar code module to carry information and instruction related to codes for the culture plate and a biological sample used in the said instrument;
an irradiation control module to carry information and instruction for an irradiation unit of the said instrument;
a sample management module to carry information and instructions for sample receiving unit of the said instrument;
an inoculation module to carry information and instructions for inoculation and streaking unit of the said instrument; and
a movement control module to carry information and instructions for various different movements occurring within the said instrument.

16. The system according to claim 15, further comprising;
a user control module to input an information regarding an experiment.

17. The system of claim 15, further comprising:
a wireless module to run the said instrument through a wireless mode.

18. The system of claim 16, wherein the user control module works in coordination with a plate dispenser module, a media dispenser module, an incubator control module, a bar code module, an irradiation control module, an inoculation module and a movement control module.
